(19) United States
(12) Patent Application Publication
(10) Pub. No.: US 2002/0177217 A1
(43) Pub. Date: Nov. 28, 2002

(54) Method for studying protein-protein interactions

(75) Inventors: Monty Krieger, Needham, MA (US); Kevin McKernan, Marblehead, MA (US)

Correspondence Address: Choate, Hall & Stewart, Exchange Place, 53 State Street, Boston, MA, 02109, US

(73) Assignee: Massachusetts Institute of Technology

(21) Appl. No.: 10/105,817

(22) Filed: Mar. 25, 2002

Related U.S. Application Data
(60) Provisional application No. 60/278,838, filed on Mar. 26, 2001.

(51) Int. Cl. C12N 1/20

(52) U.S. Cl. 435/252.3

(57) ABSTRACT

The present invention provides reagents, kits and methods for identifying and characterizing interactions between proteins and/or polypeptides. The inventive system and methods allow analysis of these interactions in vivo in eukaryotic systems, including mammalian systems. Advantages provided by various embodiments of the inventive system and methods as compared with other systems and methods for analyzing interactions between proteins or polypeptides, such as the yeast two-hybrid system, include (i) reduced ambiguity associated with identification of a potential interaction; (ii) ability to identify or study interactions that occur outside the nucleus; (iii) absence of reliance on reporter genes; and/or (iv) ability to study interactions with polypeptides that have transcriptional activation activity.

### BACKGROUND

Protein-protein interactions are involved in almost every cellular process in living cells. Therefore, elucidating protein function is an important step toward understanding the mechanisms underlying biological pathways. Furthermore, the development of therapies for the treatment of human diseases and disorders depends upon the understanding of protein function in biological processes related to the disease or disorder. In addition, with the near completion of the human genome sequencing project, the number of proteins identified with unknown function will increase dramatically. To elucidate a protein's function, it is useful to identify the interactions of a protein with other proteins.

One widely used method to study protein-protein interactions in vivo is called the two-hybrid method (Fields and Song. Nature 340:245-6, 1989; U.S. Pat. No. 5,667,973; Mendelsohn and Brent. Curr Opin Biotechnol. 5(5):482-6, 1994). The two-hybrid method relies on the in vivo activation of a reporter gene in the yeast Saccharomyces cerevisiae to detect interactions between proteins and/or polypeptides. In this artificial yeast system, a reporter gene is constructed to contain a promoter that is activated by the interaction of two fusion proteins. One fusion protein contains a DNA-binding domain (DBD), which binds to an operator positioned upstream of the promoter, and is fused to a polypeptide of interest (“bait”). The second fusion protein contains an acidic transcription activation domain (AD) which is fused to a second polypeptide of interest (“prey”). When the DBD binds to its operator, the bait becomes localized in the promoter region. If the bait and prey interact, that interaction also localizes the AD in the promoter region so that the reporter gene is turned on. If the bait and prey do not interact, the reporter gene remains silent.

By using DNA libraries to express bait and/or prey polypeptide portions of the fusion proteins, the yeast two-hybrid system allows researchers to screen a library of potential prey molecules to identify those that interact with a bait of interest (Chien et al. Proc Natl Acad Sci USA 1991 Nov 1;88(21):9578-82; Yang et al. Nucleic Acids Res 1995 Apr 11;23(7):1152-6). In addition, libraries of proteins can be screened against each other to identify novel protein-protein interactions.

However, technical disadvantages of the yeast two-hybrid limit the power of the assay. First, yeast transformants may contain multiple bait fusion expression plasmids and multiple prey fusion expression plasmids, which leads to ambiguity when a positive protein-protein interaction (“hit”) is detected because it is unclear which particular bait-prey interaction responsible for activation of the reporter gene. Second, this assay cannot be used to identify ligands that interact with a bait polypeptide that itself has some transcriptional activation activity. Third, the protein-protein interactions of interest must occur in an environment containing DNA due to the use of DNA-based reporter genes. The presence of negatively-charged DNA may affect protein-protein interactions. Fourth, protein-protein interactions in the two-hybrid assay must occur in the nucleus to be detected by the reporter gene. The bait-DBD and prey-AD fusion proteins must be able to enter the nucleus from the cytoplasm where the hybrid fusion proteins are synthesized. Moreover, any factors or additional proteins necessary that affect a protein-protein interaction of interest must be found in the nucleus to affect the interaction.

Therefore, there is a great need for methods that rapidly and accurately identify protein-protein interactions as a step towards designing therapeutic drugs and treatments for diseases.

### SUMMARY

The present invention provides an improved system and methods for identifying and characterizing protein-protein interactions. The inventive system and methods allow analysis of these interactions in vivo in eukaryotic systems, including mammalian systems. Advantages provided by various embodiments of the inventive system and methods as compared with other systems and methods for analyzing protein-protein interactions, such as the yeast two-hybrid system, include for example (i) increased certainty associated with identification of interactions; (ii) ability to identify or study interactions that occur outside the nucleus; (iii) absence of reliance on reporter genes; and/or (iv) ability to study interactions with polypeptides that have transcriptional activation activity.

In one aspect, the present invention provides methods, compositions and kits for assaying interactions between proteins and polypeptides to identify novel protein-protein interactions and/or to characterize known and novel protein-protein interactions. For example, the present invention provides a method of assaying protein-protein interactions in a collection of eukaryotic cells where individual cells express a single polypeptide-polypeptide combination to be assayed. The present invention therefore may be used to screen libraries of polypeptides/proteins (“prey”) to identify those that interact with a polypeptide/protein of interest (“bait”). The present invention may also be utilized to screen a library of proteins to identify novel protein-protein interactions encoded by the library. The present invention may also be used to screen a library of proteins against a different library of proteins to identify and characterize known and novel protein-protein interactions.

Additionally, the present invention may be used to characterize known and novel protein-protein interactions under a variety of chemical, genetics, nutritional and environmental conditions. For example, the effects of molecules or chemicals that enhance or disrupt protein-protein interactions may be assayed. Also, protein-protein interactions may be assayed in cell lines with different genetic backgrounds such as the presence or absence of oncogenes.

In certain preferred embodiments of the present invention, nucleic acids encoding bait and prey proteins are introduced into a host cell line by viral transfection. The conditions of transfection allow the introduction of genes into a collection of host cells such that individual cells express a single bait-prey polypeptide combination. The bait and prey polypeptides can encoded by a library. Alternatively or additionally, the bait polypeptide can be a particular polypeptide of interest, and the prey polypeptide encoded by a library. Also alternatively or additionally, both the bait and prey polypeptides can be particular polypeptides of interest. The nucleic acids containing genes encoding bait and prey proteins are transfected into the cells using viral-mediated nucleic acid transfer at a low multiplicity of infection resulting in a collection of cells such that individual cells express a single bait-prey polypeptide combination, thereby facilitating identification of interacting bait-prey polypeptides whenever a potential interaction is detected.

In particularly preferred embodiments of the invention, retroviral-mediated nucleic acid transfer is used to introduce nucleic acids encoding polypeptides and proteins to be assayed for interactions into a collection of host cells. Retroviral-mediated transfection allows the production of a collection of host cells where individual cells express a single polypeptide-polypeptide combination (“bait-prey”) to be assayed for an interaction. Preferably, bait and prey polypeptides to be assayed for interactions are expressed from a single transcript molecule. Alternatively or additionally, it is also preferred that genes encoding the bait and prey polypeptides are engineered to provide for comparable levels of expression of bait and prey proteins, regardless of whether one or both of the genes is integrated into the cell genome. Most preferably, bait and prey polypeptides to be assayed for interactions are translated from a single transcript molecule containing one or more internal ribosome entry site (IRES) sequences to translate additional coding sequences present in the transcript.

In another aspect, the present invention provides compositions, reagents and kits for identifying and/or characterizing protein-protein interactions utilizing a reporter system to detect an interaction between a bait polypeptide and prey polypeptide in a cell. In a preferred embodiment, compositions of the present invention include expression plasmids encoding bait and prey polypeptides, libraries and reporters constructed in accordance with the present invention. Preferred reporter systems for use in accordance with the present invention are not reliant on reporter genes in the vicinity of the protein-protein interactions of interest. Such as system has an advantage when compared with reporter genes used in conventional two-hybrid assays in that the reporter system is unlikely to exert an effect on the protein-protein interactions of interest.

In preferred embodiments of the invention, reporter systems for detecting an interaction between the bait and prey polypeptides comprise detector polypeptides. In certain preferred embodiments, the reporter system comprises one or more detector polypeptides fused with a bait polypeptide, a prey polypeptide, or both bait and prey polypeptides. The detector polypeptide(s) produces a signal which enables the determination of a bait-prey polypeptide interaction. The signal allows one skilled in the art to determine information about the bait-prey polypeptide interactions, such as the presence of an interaction, the level of polypeptides, the cellular localization of the interaction, the approximate distance separating the detector polypeptides, and the strength of the bait-prey interaction.

In a particularly preferred embodiment, a reporter system comprises detector polypeptide(s) that produces a signal where the properties of the signal are dependent on the presence or absence of an interaction between bait and prey polypeptides. For example, retroviral expression vectors may be engineered such that detector polypeptides are fused to either a bait or a prey polypeptide or both bait and prey polypeptides. The vectors are expressed in host cells in accordance with the present invention and the bait and prey polypeptides are assayed for an interaction. The presence of an interaction between bait and prey polypeptides results in a signal produced by the detector polypeptide(s) that is detectably different than the signal produced in the absence of an interaction.

In another preferred embodiment, a reporter system comprises a detector polypeptide that produces a signal and a localization entity (preferably a polypeptide or complex of polypeptides) that localizes a bait and/or prey polypeptide within the cell, preferably within a sub-cellular compartment. The detector polypeptide is fused to one member of a bait-prey pair, and the localization polypeptide is fused to the other member of the bait-prey pair. The fusion proteins are expressed in a collection of host cells in accordance with the present invention, and interactions between bait and prey polypeptides are assayed. In the presence of a bait-prey interaction, the detector polypeptide is sequestered within the cell by the localization molecule through the bait-prey interaction, and as a result, a signal is produced inside the cell indicating a bait-prey interaction. By contrast in the absence of a bait-prey interaction, the detector polypeptide is not sequestered within a cell by the localization polypeptide through bait-prey interactions, and is preferably secreted from the cell.

In another preferred embodiment, a reporter system comprises a detector polypeptide that produces a signal, and is fused to either a bait or a prey polypeptide. The reporter system further comprises a cleaving molecule such as a protease fused to the other polypeptide of the bait-prey combination, and acts to detach the detector polypeptide from the fusion protein when the bait and prey interact. The fusion proteins are expressed in a collection of host cells in accordance with the present invention and interactions between bait and prey polypeptides are assayed. In the absence of a bait-prey interaction, the cleaving molecule is unable to separate the detector polypeptide from its fusion protein. Thus, the detector polypeptide remains as part of the fusion protein and therefore produces a signal. In the presence of a bait-prey interaction, the cleaving molecule detaches the detector polypeptide from its fusion protein, releasing the detector polypeptide, which is preferably secreted from the cell. Therefore, a bait-prey interaction is detected by the absence of the signal produced by the detector polypeptide in the cell.

In another preferred embodiment, the reporter system comprises a polypeptide that activates or represses a biological process, and optionally further comprises a localization polypeptide. Such biological processes include without limitation, transcription of a reporter gene, signal transduction, apoptosis, DNA replication, formation of cellular macrostructures (such as the cytoskeleton, nuclear scaffold, mitotic spindle, nuclear pores, centrosomes, and kinetochores), enzymatic processes (such as proteases and kinases), senescence, cell cycle arrest, cell cycle checkpoints, secretion of proteins and molecules, metabolic pathways, translation of RNA, resistance to antibiotics, resistance to viral infection, resistance to chemicals, temperature sensitivity, tolerance or sensitivity to environmental conditions (such as pH, light, radiation, magnetism, desiccation, ionic strength, and pressure), sensitivity or tolerance to cell-cell contact, auxotrophic requirements, endocytosis, and binding of ligands to receptors.

In this embodiment, the activation/repression detector is preferably fused to one of the polypeptides of the bait-prey combination. In the absence of a bait-prey interaction, the activation/repression detector is not sequestered and therefore functions to activate or repress the pathway of interest. In the presence of a bait-prey interaction, the activation/repression detector is sequestered and unable to activate or repress the pathway of interest thereby allowing the detection of a bait-prey interaction. Particularly preferred biological pathways are transcription of a reporter gene at a distance far removed from the bait-prey interaction, and activation of a signal transduction pathway leading to the detectable phosphorylation of a protein.

In yet another aspect, the present invention provides a collection of eukaryotic host cells, each of which expresses a bait polypeptide of interest and also expresses a prey polypeptide of interest. The cells further contain a reporter system to allow detection of an interaction between the bait and prey polypeptides. The present invention also provides vectors and viral lines which allow transfection of nucleic acids into the host cells such that, at most, single polypeptidepolypeptide combinations are expressed in each host cell.

In yet another aspect, the present invention provides reagents and kits comprising viral expression vectors, viral lines, nucleic acid libraries, systems for producing viral particles containing coding sequences for polypeptides to be assayed for interactions, reporter systems, host cell lines and combinations thereof to practice the present invention.

### Definitions

“Peptide”. The term “peptide” is used herein to mean at least two amino acids that are covalently linked with a peptide bond. A peptide bond is commonly known in biochemistry as an amide linkage between the carboxyl group of one amino acid and the amino group of another. Preferred sizes of peptides range from 2 amino acids to 20. Particularly preferred sizes of peptides range from 3-15 amino acids. Generally peptides having at least 3 amino acids have a linear amino acid sequence. For example, one amino acid is linked through a peptide bond to a second amino acid. A third amino acid is linked to the second. Preferably, a peptide as used herein does not have secondary structure.

“Polypeptide”: The term “polypeptide” is used herein to mean at least two amino acids that are covalently linked with a peptide bond. Preferred sizes of polypeptides peptides range from 2 amino acids to several thousands of amino acids. Particularly preferred sizes of polypeptides range from 20 to several hundred amino acids. Therefore a peptide is polypeptide.

“Protein”: The term “protein” is used herein to mean a polypeptide that is capable of performing a biological function. Proteins can range in size from approximately 10-15 amino acids to several thousand amino acids in length. As used herein, all proteins are polypeptides and the terms may be used interchangeably.

“Detectors”: Detectors of the present invention as used herein are reporter molecules is including proteins and polypeptides that provide a detectable signal to enable one of ordinary skill in the art to determine information regarding an interaction between a bait polypeptide and a prey polypeptide such as the presence of an interaction, the level of polypeptides, the cellular localization of the interaction, the approximate distance separating the detector polypeptides, and possibly the strength of the bait-prey interaction.

“Bait and prey polypeptides/proteins”: Bait and prey polypeptides/proteins as used herein refer to the proteins or polypeptides to be screened for a physical interaction

“Fusion protein”: A fusion protein as used herein refers to a protein formed by the expression and translation of a hybrid (or chimeric) gene constructed by combining two gene sequences in frame with each other.

“Nucleic acid molecule”: A nucleic acid molecule as used herein refers to deoxyribonucleic acids (DNA), and ribonucleic acids (RNA), including messenger RNA (mRNA) and transfer RNA (tRNA). Nucleic acids comprise a phosphate backbone, a fucose sugar moiety and a nitrogenous bases. Nucleic acid molecules are single stranded, double stranded, and also tripled stranded. A double stranded nucleic acid may comprise two single strands of nucleic acid molecules hybridized to each other through hydrogen bond-mediated base pairing hybridization. A double stranded nucleic acids may also comprise two regions of a one nucleic acid molecule that hybridize to each other to form secondary structure.

“Expression vectors”: Expression vectors as used herein are nucleic acid molecules that direct the transcription of DNA to mRNA by RNA polymerase of a coding sequence of interest. Expression vectors are also nucleic acid molecules that direct the translation of mRNA to proteins. Expression vectors are also RNA molecules that direct the reverse transcription of RNA to DNA by a reverse transcriptase. Expression vectors may be single-stranded or doublestranded. Expression vectors may be circular or linear molecules.

“Transformation and transfection”: Transformation and transfection as used herein referred to the introduction of nucleic acid molecules into a host cell. In general, transformation of nucleic acids involve the introduction of nucleic acids into a host cell without integration into the host cell genome. Generally, transformation of nucleic acids into bacterial and yeast cells are performed by standard techniques in molecular biology such as electroporation, heat shock, and calcium phosphate. Transfection of nucleic acids into a host cell involves the introduction and integration of nucleic acids into host cell genomes of eukaryotic cells. Methods of transfection are well known in the art and include without limitation, viral mediated transfection, electroporation, particle bombardment, and calcium phosphate-mediated.

“Collection”: A collection as used herein when referring to a collection of cells means a collection of cells from the same cell line. Preferably the collection of cells contains cells which are descendants from a single clone or cell.

“Assay”: The term “assay” as used herein refers to the identification and/or characterization of known or novel protein-protein and polypeptide-polypeptide interactions.

“Set or combination”: A “set” or “combination” of proteins as used herein refers to a specific group of proteins that interact or that are being assayed for interactions. The terms are used interchangeably.

### DETAILED DESCRIPTION OF CERTAIN PREFERRED EMBODIMENTS

The present invention relates to a novel system and methods for analyzing and detecting interactions between proteins and polypeptides in eukaryotic cells. In some embodiments, the present invention provides a method for identifying and characterizing known and novel proteinprotein interactions by providing a collection of eukaryotic host cells where each cell expresses, at most, a single “bait-prey” polypeptide-polypeptide combination. Bait and prey polypeptides may be particular polypeptides of interest and/or members of a protein library encoded by a nucleic acid library and expressed in the collection of cells. Therefore, as apparent to one of ordinary skill, a bait polypeptide may be a particular polypeptide sequence of interest which may be screened against a library of prey polypeptides to identify novel interactions. Also, a bait polypeptide of interest may be assayed for an interaction with a prey polypeptide of interest under a variety of chemical, genetic and environmental conditions which may increase or decrease the affinity of the bait polypeptide for the prey polypeptide. Furthermore, a library of bait polypeptides may be screened against a library of prey polypeptides to identify and characterize known and preferably novel protein-protein interactions. It is recognized that multiple proteins may be involved in interactions with each other. For example, three or more proteins may be involved and also may be necessary to form a complex. Such higher order complexes are within the scope of the present invention and may be identified and analyzed by the present invention.

Methods of the present invention have certain advantages, including (1) the ability to study protein-protein interactions inside and outside the nucleus, and optionally, within a sub cellular organelle of interest; (2) detection of protein-protein interactions without relying on reporter genes near the location of the protein-protein interaction of interest; and (3) study of interactions with polypeptides that have transcriptional activation or repression activity.

Conventional two-hybrid technology in yeast relies on the transcriptional activation of a reporter gene to detect the presence of a protein-protein interaction of interest (Fields and Song. Nature 1989 July 20;340(6230):245-6). More specifically, the two-hybrid assays utilize the two modular domains of a transcription factor, namely the DNA-binding domain and the acidic activation domain, as tools to detect protein-protein interactions. The two domains are separated and individually fused to polypeptides of interest. The DNA-binding domain (DBD) is fused to the first polypeptide or protein (“bait”) and anchors the bait protein to the promoter region of a reporter gene. The activation domain (AD) is fused to the second polypeptide or protein of interest (“prey”) and activates transcription of the reporter gene when the prey protein physically interacts with the bait protein. The result is a detection system that only activates transcription of a reporter gene when a protein-protein interaction between the bait and the prey recruits the activation domain to the promoter to activate transcription of the reporter gene.

Fotin-Mleczek et al. (Biotechniques 29:22-26, July 2000) and Luo et al. (U.S. Pat. No. 6,114,111) have extended the use of the two-hybrid system of Fields and Song to mammalian cells. The basic approach developed by Fields and Song (Nature 1989 July 20;340(6230):245-6) is applied to mammalians cells. Fotin-Mleczek et al. (supra) and Luo et al. (supra) both describe the use of a DNA-binding domain fused to a “bait” protein and a transcriptional activation domain fused to a “prey” protein to detect bait-prey protein-protein interactions. As with the yeast two-hybrid method, the detection of a bait-prey interaction is facilitated by a DNA reporter gene which is activated by the transcriptional activation domain of the prey-AD fusion protein.

Since the two-hybrid technology in yeast (Fields and Song, supra) and mammalian systems (Fotin-Mleczek et al., supra; Luo et al., supra) utilizes DNA-based reporter genes for detection, protein-protein interactions of interest must occur close to the promoter of the DNA reporter. Without limitation to theory, several problems resulting from the presence of DNA can adversely affect the protein-protein interactions being studied. For example, counterion condensation around the negatively charged DNA results in high ionic strength around the DNA. High ionic strength is known to reduce electrostatic interactions between molecules such as polypeptides and nucleic acids. DNA may also sterically hinder one protein from binding to the another protein.

Other disadvantages limit the power of the conventional two-hybrid system. For example, protein-protein interactions must occur in the nucleus to be detected by activation of the transcriptional reporter. Therefore, proteins of interest being assayed for an interaction must enter the nucleus or otherwise have a nuclear localization domain which might affect the interactions. In addition, any necessary co-factors or accessory proteins needed for the protein-protein interaction of interest to occur must be found in the nucleus.

Another disadvantage with the conventional two-hybrid system is that transformation or transfection of expression vectors from a library may result in the expression of multiple members of the library in a single cell. The result is that multiple bait-prey interactions are possible in one cell producing ambiguity when a positive bait-prey interaction is detected (“hit”). Thus, a screen for protein-protein interactions using the conventional two-hybrid assay may include the additional task of pinpointing the exact bait-prey interaction responsible for the activation of the reporter gene. This ambiguity adds to the time and number of steps necessary before a protein-protein interaction is determined.

False activation of the reporter gene poses yet another problem for screens using the conventional two-hybrid system. For example, the fusion protein containing the DNA-binding domain and a “bait” polypeptide, in the absence of the AD-prey fusion protein, may activate transcription of the reporter gene. Moreover, the AD-prey fusion protein may activate transcription of the reporter by binding directly to the promoter of the reporter. Furthermore, mutations can occur in yeast and mammalian cells that result in transcriptional activation of the reporter without a protein-protein interaction between the bait and prey domains of the hybrid proteins.

The present invention provides methods and compositions for studying interactions between proteins and polypeptides which are more versatile than conventional methods such as the two-hybrid method. In one aspect, the present invention provides a method of identifying novel protein-protein interactions and characterizing known and novel protein-protein interactions in eukaryotic systems such as mammalian systems. Protein-protein interactions can be assayed according to the present invention inside and/or outside the nucleus, and preferably within a sub-cellular location of interest. Protein-protein interactions are also assayed in the absence of a reporter gene which is closely located to the protein-protein interaction of interest which may negatively influence the interaction. In addition, the absence of a reporter gene close to the protein-protein interaction of interest allows proteins with transcriptional activation and repression activity to be assayed for interactions with other proteins.

Furthermore, the present invention assays protein-protein interactions in a collection of host cells such that individual cells express a single “bait-prey” protein-protein combination to be assayed. Expression of a single bait-prey combination in individual cells reduces the ambiguity associated with identification of a potential bait-prey interaction. The reduction in ambiguity is especially useful when a polypeptide of interest (bait) is screened against a library of potential interactors (prey) or for screening a library of potential interactors with another library of potential interactors. Conventional screening methods may result in multiple components of a library in each cell. In these cases, identification of a bait-prey interaction requires further experimentation to determine which member of the library is interacting with the bait.

In a preferred embodiment of the present invention, viral-mediated nucleic acid transfer is used to transfect one or more libraries of genes into a collection of cells at a low multiplicity of infection (MOI) resulting in individual cells that express a single polypeptide-polypeptide combination (bait-prey) to be assayed. Therefore, the present invention is useful for screening libraries of polypeptides/proteins for potential interactions with a polypeptide/protein of interest. The present invention is also useful for screening libraries of polypeptides/proteins for potential interactions with other polypeptides/proteins encoded by another (or same) library.

It is recognized that conventional methods of plasmid transformation and transfection which result in a majority of cells transformed or transfected with at most a single plasmid molecule may be used in accordance with the teachings of the present invention. A non-limiting example includes the transformation or transfection of a library of plasmids using low concentrations of plasmids and cells to limit the number of cells transformed or transfected with multiple plasmids.

The present invention takes advantage of the ability of a single viral particle (virion) to transfect a cell with a nucleic acid molecule contained within the virus. Under conditions of a low multiplicity of infection (MOI), one skilled in the art using known methods can control the average and maximum number of viruses that infect individual cells in a collection of host cells. Each virion may contain a single coding sequence of a protein to be assayed. Therefore a library may be contained with the collection of viruses. Cells may be transfected at a low MOI such that individual cells are singly transfected or doubly transfected to express a single bait-prey pair combination from the one or more libraries.

In a particularly preferred embodiment of the present invention, viral-mediated nucleic acid transfer is used to transfect a single nucleic acid molecule into a cell which contains the coding sequences for both the bait and the prey polypeptides to be assayed. Viral expression vectors are constructed containing multiple cloning sites for insertion of multiple coding sequences. Libraries of genes can therefore be encoded by a collection of expression vectors, where each vector molecule contains the coding sequences of a single bait-prey polypeptide combination. Either or both of the bait and the prey may be components of libraries. Alternatively, the bait is a polypeptide of interest and the prey is component of a libraries. Additionally for certain embodiments to characterize a protein-protein interaction, both the bait and the prey are a polypeptide of interest. Therefore, a single bait-prey polypeptide combination can be expressed in individual cells by introducing one vector molecule into individual cells.

IRES

In a particularly preferred embodiment, single bait-prey polypeptide/protein pair combinations are expressed in a host cell from a single nucleic acid molecule using a retroviral expression vector containing a promoter, multiple coding sequences and at least one internal ribosome entry site (IRES). The promoter is used to activate transcription of the coding sequences and the IRES. Any promoter that is active in the relevant expression system may be used in accordance with the present invention. Numerous promoters are known to those skilled in the art. Preferably, the promoter is a viral promoter. These promoters include without limitation constitutive promoters, inducible promoters, viral LTR (long terminal repeat) promoters, CMV promoter (cytomegalovirus), RSV promoter (Rous sarcoma virus), SV40 promoter, cauliflower mosaic viral (CaMV), Vlambdal promoter, and EF1 alpha.

Transcription of the expression vector produces mRNA molecules having a first coding sequence, an IRES and at least one additional coding sequence. An IRES positioned 5′ to additional coding sequence directs the co-translation of additional multiple open reading frames (ORF) from a single polycistronic RNA message (for a review see Martinez-Salas. Current Opinion in Biotechnology. 10:458-464, 1999). Briefly, IRES are cis-acting elements that recruit the small ribosomal subunits to an internal initiator codon in the mRNA with the aid of cellular trans-acting factors (Martinez-Salas. supra). A polycistronic message having correctly positioned IRES sequences directs the co-translation of multiple ORFs in a polycistronic mRNA. IRES sequences have previously been used to co-express two genes where one gene is a selectable marker (or a reporter gene) and the other gene encodes a protein of interest. Co-expression of the two genes and subsequent selection ensures the co-expression of the protein of interest (Liu et al. Anal Biochem. 280(1):20-8, Apr. 10, 2000; Zhu et al. Cytometry. 37(1):51-9, Sept. 1, 1999; Aran et al. Cancer Gene Ther. 5(4):195-206, 1998; Levenson et al. Hum Gene Ther. 9(8):1233-6, May 1998; and U.S. Pat. No. 5,968,738 to Anderson et al.).

Preferably, the expression vector transcripts are packaged in a retrovirus with each virion containing one nucleic acid molecule. The packaging is facilitated by a “packaging cell line.”Any cell line capable of expressing a viral vector and a second vector encoding viral particles to produce viruses containing nucleic acids expressed from the viral vector may be used as packaging cell lines. Various packaging cell lines are available. A commonly used packaging cell line is the Phoenix retroviral packaging cell line (American Type Culture Collection, ATCC SD 3444). Additional packaging cell lines are available from Clontech Laboratories (Palo Alto, Calif.) which are based on the HEK 293 or NIH 3T3 cell lines. In a preferred method of packaging vectors into viral particles, the expression vector and a second vector encoding viral proteins are transfected into the host packaging cell line using non-viral mediated methods such as electroporation and calcium phosphate method (Chatterton et al. Proc. Natl. Acad. Sci. USA 96:915-920, 1999; Ausubel et al. “Current Protocols in Molecular Biology” Wiley & Sons. 1999 incorporated herein by reference). The expression vectors encoding test proteins produce mRNA molecules in the packaging cells which are packaged into viral particles by the viral proteins encoded by the second vector. Each viral particle thus contains one RNA molecule transcribed from an expression vector encoding a single bait-prey combination of proteins. The viral particles are harvested and used to infect a target cell line at a low MOI such that individual cells are not infected by more than one virus particle (virion).

Retroviral vectors and expression systems are readily available from commercial sources (see for example, Clontech Laboratories, Palo Alto, Calif.; Promega Corporation, Madison, Wis.; Invitrogen Corporation, Carlsbad, Calif.; and IMGENEX, San Diego, Calif.). Such systems include the retroviral expression vector with suitable cloning sites, a vector expressing viral particles, packaging cell lines, and cDNA expression libraries. In addition, expression vectors containing IRES sequences are known to those skilled in the art (see Clontech Laboratories). Examples of retroviral expression vectors available from Clontech include pLEGFP-N1, pLEGFP-C1, pLXIN, pSIR, pLXSN, pLNCX, and pLAPSN. Furthermore, IRES containing expression vectors are available from Clontech such as pIRES, pIRES-EGFP and pIRES-EYFP.

It is recognized that the expression of multiple polypeptides from a single DNA retroviral vector/construct can utilize multiple promoters, multiple transcriptional start and stop sequences, and multiple translational start and stop sequences to produce multiple mRNA molecules rather than using an IRES which directs translation of multiple polypeptides from a single mRNA message. However, the use of the IRES sequence is preferred due to the smaller size of the IRES sequence as compared with the use of multiple promoter sequences. It is preferable to reduce the size of the expression vector by using IRES sequences which allows the vector to accommodate large coding sequence.

Viruses

As discussed herein, preferred expression and transfection systems for use in accordance with the present invention are viral systems. Any virus that can transfer nucleic acids into a host cell may be used in accordance with the present invention. In general, these viruses include mammalian type viruses, adenoviruses, retroviruses, baculoviruses, plant viruses. Retroviruses are preferred for use because reverse-transcribed DNA is stably integrated into the host genome. A list of suitable retroviruses is available in “Retroviruses.” (ed. J. M. Coffin et al., Cold Spring Harbor Laboratory Press, Cold Spring Harbor, N.Y. 1997 incorporated herein by reference.) A particularly well suited retroviral transfection system is described in Mann et al. (Cell 33:153-159, 1993); Pear et al. (Proc. Natl. Acad. Sci. USA 90(18):8392-6, 1993; Kitamura et al. (Proc. Natl. Acad. Sci. USA 92:9146-9150, 1995); Kinchella et al. (Human Gene Therapy 7:1405-1413, 1996); Hofmann et al. (Proc. Natl. Acad. Sci. USA 93:5185-5190, 1996); Choate et al. (Human Gene Therapy 7:2247, 1996); WO 94/19478; PCT US97/01019, and references cited therein, all of which are incorporated by reference.

Other references for lists of suitable retroviruses for use in the present invention include the National Center for Biotechnology Information at the National Institutes of Health (Bethesda, Md.), and the American Type Culture Collection (ATCC, Manassas, Va.). The NCBI website includes a link to resources for researchers utilizing retroviruses:

(http://www.ncbi.nlm.nih.gov/retroviruses/)

Cells

Any eukaryotic cell line that can be transformed or transfected with nucleic acid constructs according to the teachings of the present invention may be used. Preferred eukaryotic cells can be transfected with a virus. More preferred are cells that can be transfected with a retrovirus and contain cell surface receptors for the retrovirus. One of ordinary skill also can generate a cell line that expresses the retrovirus receptor by transfecting the cell line with a plasmid coding for the receptor (Gu et al. supra).

Eukaryotic cells for use in the present invention include but are not limited to mammalian cells, plant cells, insect cells, worm cells, fish cells, avian cells, reptilian cell and arthropod cells. Commonly studied systems include without limitation Drosophila, C. elegans, zebra fish, Xenopus, numerous plants such as Arabidopsis, tobacco, corn. For a comprehensive list of cell lines available to researchers, see the American Type Culture Collection catalogue (ATCC; Manassas, Va.; incorporated herein by reference). A list of suitable plant cells is available from the DSMZ German deposit (Deutsche Sammlung von Mikroorganismen und Zellkulturen GmbH, Braunschweig, Germany) and the ATCC.

Mammalian cells are the preferred host cells for use with the present invention. Those skilled in the art of using mammalian cell lines will recognize that a wide variety of mammalian cell lines may be used in accordance with the present teachings. Preferred mammalian cell lines are derived from humans, rats, mice, rabbits, monkeys, hamsters, and guinea pigs since cells lines from these organisms are well-studied and characterized. However, the present invention does not limit the use of mammalians cells lines to only the ones listed.

Suitable mammalian cell lines are often derived from tumors. For example, the following tumor cell-types may be sources of cells for culturing cells: melanoma, myeloid leukemia, carcinomas of the lung, breast, ovaries, colon, kidney, prostate, pancreas and testes), cardiomyocytes, endothelial cells, epithelial cells, lymphocytes (T-cell and B cell), mast cells, eosinophils, vascular intimal cells, hepatocytes, leukocytes including mononuclear leukocytes, stem cells such as haemopoetic, neural, skin, lung, kidney, liver and myocyte stem cells (for use in screening for differentiation and de-differentiation factors), osteoclasts, chondrocytes and other connective tissue cells, keratinocytes, melanocytes, liver cells, kidney cells, and adipocytes. Non-limiting examples of mammalian cells lines that have been widely used by researchers include HeLa, NIH/3T3, HT1080, CHO, COS-1, 293T, WI-38, and CV-1/EBNA-1. For an extensive list of mammalian cell lines, those of ordinary skill in the art may refer to the American Type Culture Collection catalogue.

The selection of the host cell line may take into consideration the post-translational processing in a particular cell line. For example, when studying protein-protein interactions from humans, it may be desirable in the present invention to use a human host cell line so that bait and prey proteins are translated and modified post-translationally in accordance with their native environment.

Detection

In another aspect of the present invention, a reporter system is used to detect an interaction between a bait and a prey polypeptide expressed in a host cell in accordance with the present invention. In a particularly preferred embodiment of the invention, the reporter system comprises one or more detector polypeptides which produce a detectable signal to indicate the presence or absence of an interaction between a bait and prey polypeptide. Detectable signals produced by the detector polypeptides include but are not limited to light, fluorescence, luminescence, bioluminescence, chemiluminescence, enzymatic reaction products, electricity, electric potential, magnetism, radiation, color, and chemicals. In addition, a detectable signal includes detectable alterations in biological pathways and responses.

In one preferred embodiment, the detectors are polypeptides which are fused to the bait polypeptide and/or the prey polypeptide to form fusion proteins. The detector polypeptides of the present invention are designed to produce a signal when an interaction between a bait polypeptide and a prey polypeptide is present in a cell. The signal should be detectably different than a signal produced in the absence of a bait-prey interaction, should allow one skilled in the art to identify cells containing a bait-prey interaction. It is recognized that a detectable signal in the presence of a bait-prey interaction can be greater than or less than the signal in the absence of a bait-prey interaction.

Fluorescence Resonance Energy Transfer (FRET)

In a preferred embodiment, a reporter system comprises polypeptide-based fluorescent proteins as detector polypeptides. In the present embodiment, the fluorescent detection polypeptides are fused to bait and prey polypeptides to produce fusion proteins. The fluorescent fusion proteins are encoded by expression vectors and expressed in host cells according to the teachings of the present invention. Preferably, the coding sequence of a detection polypeptide is positioned in frame with the coding sequence of a test protein for proper translation of the fusion protein. The detection polypeptide may be fused either to the N-terminal end or the C-terminal end of the test protein.

When expressed in a cell in accordance with the present invention, the bait-fluorescent fusion protein and the prey-fluorescent fusion protein are assayed for a physical interaction by using fluorescent resonance energy transfer (FRET). FRET is a method of detecting protein-protein interactions by utilizing the transfer of energy from an excited donor molecule to an acceptor molecule through dipole-dipole coupling. The efficiency of energy transfer resulting in a detectable signal is directly proportional to the distance separating the donor and acceptor, and the spectral overlap between the fluorescence emission spectrum of the donor emission and absorption spectrum of the acceptor molecule. (Pollock and Heim, Trends in Cell Biol. 9:57-60, February 1999; Bastiaens and Squire. Trends in Cell Biol. 9:48-52, February 1999, both references are incorporated herein in their entirety).

Detection of FRET uses a donor molecule's photophysical or photochemical properties. Typically, excitation of the donor produces light which is absorbed by the acceptor to produce excited light from the acceptor molecule. Therefore, a detectable signal in methods using FRET comprises the light emitted from the acceptor molecule and/or the decrease in the amount of light emitted from the donor molecule due to the absorption by the acceptor.

Examples of fluorescent proteins includes without limitation a green fluorescent protein derived from the jellyfish Aequorea Victoria, and luciferase derived from the firefly (Photinus pyralis) or the sea pansy (Renilla reniformis) and mutants thereof. In a preferred embodiment of a polypeptide-based detection system, the green fluorescent protein (GFP) from the jellyfish Aequorea victoria is used as a polypeptide-based detection method. Briefly GFP (and its derivatives which have varying color and intensity; Pollok and Heim, supra) is a protein which fluoresces when excited by an excitation source and without the use of co-factors. In recent years, mutants of GFP have been discovered which have different emission and excitation spectra.

The selection of GFPs to use in the present invention should take into consideration several factors. First, the excitation spectra of the GFP proteins should be sufficiently separated to enable one to selectively excite different GFPs. In addition, the emission spectrum of the donor should overlap the excitation spectrum of the acceptor to maximize energy transfer. Furthermore, the emission spectra of different GFPs should be sufficiently separated to enable one to distinguish the GFP molecules that are fluorescing. Those of ordinary skill in the art can readily selected GFPs to use in accordance with the present invention based on well characterized GFPs in the art. However, the present invention is not limited to only the GFPs described herein and also are not limited to GFPs and mutants known in the art. For example, new GFP mutants may be discovered which may be used in the present invention and are included by the scope of the present invention.

Classes of GFP mutants and use of GFPs in FRET are described in Pollock and Heim and are briefly discussed herein. The eGFPs are a class of proteins that has various substitutions of the serine at position 65 (Ser65). For example, Thr, Ala, and Gly have been used. The peak of the emission spectrum of the eGFPs is most likely close to wild-type GFP (511 nm), but has a different excitation peak. The blue fluorescent proteins (BFP) have a mutation at position 66 (Tyr to His mutation) which alters its emission and excitation properties. This Y66H mutation in BFP causes the spectra to be blue-shifted compared to the wtGFP. Cyan fluorescent proteins (CFP) have a Y66W (Tyr to Trp) mutation with excitation and emission spectra wavelengths between those of BFP and eGFP. Sapphire is a mutant with the excitation peak at 495 nM suppressed while still having the excitation peak at 395 and the emission peak at 511 nM. Yellow FP (YFP) mutants have an aromatic amino acid (e.g. Phe, Tyr, Trp) at position 203 and have red-shifted emission and excitation spectra.

To provide a descriptive non-limiting example of GFPs used in FRET, two GFP pairs are described herein and may be used in accordance with the present invention. In one pair, BFP is used as a donor molecule and eGFP as the acceptor molecule due to the spectral overlap between the BFP emission and the eGFP excitation spectra. BFP and eGFP expression vectors are both commercially available which has led to their wide use in FRET assays. For example Clontech Laboratories (Palo Alto, Calif.) has expression vectors for BFP proteins. In addition, other mammalian expression vectors for BFP are available from Quantum Biotechnologies (pQBI50-BFP; Laval, Quebec).

Additionally or alternatively, CFP and YFP may be used as a pair of fluorescent proteins for use in FRET. Other examples of GFP pairs that have been used to study protein-protein interactions are found in the following references, the teachings of which are all incorporated herein by reference in their entirety. (Day. Mol. Endocrinol. 12:1410-1419, 1998; Romoser et al. J. Biol. Chem. 272:13270-13274, 1997; Miyawaki et al. Nature 388:882-887, 1997; Mahajan et al. Nat. Biotechnol. 16:547-552, 1998)

Briefly, Day used BFP and eGFP fusion proteins in a FRET assay to detect homodimerization of the Pit-1 transcription factor in HeLa cells. Miyawaki et al. fused either BFP or CFP to the N-terminus of calmodulin and either eGFP or YFP to the C-terminus of M13 which is a calmodulin-binding peptide derived from the smooth muscle myosin light chain kinase (MLCK). Miyawaki et al., using this pair of fluorescent fusion proteins, was able to study the interactions in transiently transfected HeLa cells using DNA based transfection.

From the teachings herein, one of ordinary skill in the art of constructing expression vectors for GFP fusion proteins can readily use either IRES based vector or a vector construction using multiple promoters to express a polycistronic message encoding multiple fusion polypeptides comprising proteins encoded by a library fused to GFP. Subsequent to retroviral mediated transfection into mammalians cells under conditions of low MOI according to the teachings of the present invention, those skilled in the art can readily detect protein-protein interactions in cell-based systems using FRET and standard fluorescent detection methods such as fluorescence activated cell sorting (FACS) and fluorescence microscopy.

For constructing expression vectors, one skilled in the art may refer to commercially available sources of vectors for expression of GFP fusion proteins according to the teaching herein (e.g. Clontech, Palo Alto, Calif.; Aurora Bioscience Corporation, San Diego, Calif.). Furthermore, those skilled in the art are readily capable of constructing retroviral expression vectors containing GFP coding sequences, cloning sites upstream or downstream of the GFP coding sequences to create GFP fusions, promoters, and IRES sequences to facilitate translation of the polycistronic messages.

Those skilled in the art recognize that variations to the FRET assays described are readily appreciated based on the teachings herein. For example without limitation, one or more antibodies directed to the individual polypeptides in a protein-protein interaction of interest may be used that contains a GFP fusion or a fluorescent chromophore can act as the donor and/or acceptor molecule in the FRET assay.

Bioluminescence Resonance Energy Transfer

In another preferred embodiment, reporter systems comprise bioluminescent and fluorescent proteins, and utilize energy transfer between the proteins to detect an interaction between bait and prey proteins. In this embodiment, bioluminescence resonance energy transfer (BRET) is used to detect the interaction of bait and prey proteins expressed in vivo according to the teachings of the present invention. BRET is a naturally occurring phenomenon, and unlike FRET, does not require excitation energy from an external source. In general, BRET utilizes a bioluminescent protein which luminesces without excitation. The bioluminescent protein may or may not require a co-factor/substrate which facilitates light emission. The bioluminescent protein emits photons which are absorbed by an acceptor fluorophore if the fluorophore is in close proximity to allow resonance energy transfer. Preferably, the acceptor fluorophore is excited by the photons and fluoresces to emit light at a wavelength that is different than the wavelength of light emitted by the bioluminescent protein (see for example Xu et al., Proc. Natl. Acad. Sci. USA 96:151-156, January 1999; Angers et al., Proc. Natl. Acad. Sci. USA 97(7):3684-3689, Mar. 28, 2000) both of which are incorporated herein by references). The result is that the fluorescent protein quenches the bioluminescent and alters the wavelength of light produced by fluorescing at the different wavelength. Advantages to the use of BRET include the lack of an external excitation source which photobleaches fluorescent and bioluminescent proteins. Additionally, the lack of an external excitation source permits the study of cells that may be sensitive to light or light damage.

Bioluminescent proteins used in BRET include the luciferase from R. reniformis (R-luc). However, other bioluminescent proteins and other luciferases may be used if the emission spectrum is similar in wavelength to the excitation spectrum of the acceptor protein. The substrate for R-luc is coelenterazine which is a hydrophobic molecule which when degraded by R-luc, luminesces. R-luciferase was shown to exhibit FRET with a red-shift variant of GFP (YFP). Xu et al. studied BRET with R-luciferase and YFP in E. coli and suggested that energy transfer between the two proteins occurs when the proteins are approximately 50 angstroms apart. Angers et al. applied the BRET system to mammalians cells and demonstrated the dimerization of two proteins that were individually fused to R-luciferase and YFP. However, Xu et al. and Angers et al. are silent on the use of a low MOI to express a library of retroviruses in a collection of host cells.

Localization

In general, proteins of the present invention may be targeted to sub-cellular organelles or other cellular components using peptides that direct the intracellular transport of proteins and localize proteins within a cellular compartment. Therefore protein-protein interactions may be studied within a specific cellular environment including the nucleus, cytoplasm, mitochondria, Golgi, membranes, cell wall, endoplasmic reticulum, lysosomes, and vacuoles depending on the bait-prey proteins being assayed. For a general discussion of signal peptides see Nielsen et al. (Protein Engineering 10:1-6, 1997; the teachings of which are incorporated herein by reference) and references cited therein.

In another preferred embodiment, the reporter system comprises localization polypeptides and signal-producing polypeptides. A localization polypeptide is fused one member of the bait-prey pair, and a signal-producing polypeptide is fused to the other member of the bait-prey pair. An expressed fusion protein containing a localization polypeptide and a bait polypeptide for example, will be sequestered within the cell, and preferably within a sub-cellular organelle of interest. In the absence of a bait-prey interaction, the fusion protein containing the signal-producing polypeptide is not sequestered within the cell through the bait-prey interaction, and is preferably secreted from the cell. In the presence of a bait-prey interaction, the fusion protein containing the signal-producing polypeptide is sequestered in the cell indirectly via the fusion protein containing the localization polypeptide. As a result, a signal is produced inside the cell and is therefore detected to indicate a bait-prey interaction.

Bait and prey polypeptides can be targeted to subcellular organelles such as the endoplasmic reticulum (ER), Golgi bodies, cell wall membrane, lysosomes, and mitochondria for example. Those skilled in the art can use signal peptides to target bait and prey polypeptides to sub-cellular organelles. Signal peptides are well known in the art as referring to peptides that enable a cell to sort and target a protein to a particular sub-cellular organelles (for a general discussion see Cell 1999 Dec 10;99(6):557-8 which refers to work by G. Blobel and colleagues). Such signal peptides sequences are well known in the art and may be used in the present invention to target and localize test proteins.

As a non-limiting example of a localization protein, the temperature sensitive mutant of the vesicular stomatitis virus G protein (VSVG ts-045) is used to localize bait and/or prey polypeptides to the ER. VSVG misfolds at non-permissive temperatures, and as a result is retained in the ER (Hirschberg et al. J. Cell Biol. 143:1485-1503, 1998). In accordance with the present invention, a bait polypeptide is fused to the VSVG temperature sensitive mutant and is therefore sequestered at the ER when expressed in a cell at the non-permissive temperature. A prey polypeptide, which may be fused to a signal producing polypeptide or which may itself be a signal producing polypeptide (e.g. such as a transcription factor or a fluorescent protein) is assayed for an interaction with the bait polypeptide after being expressed in cells according to the present invention. A bait-prey interaction sequesters the signal-producing polypeptide at the ER which may be detected directly or through the activation (or repression) of a biological process. In the absence of a bait-prey interaction, the signal-producing polypeptide is not sequestered at the ER, and is either secreted from the cell or represses a biological process.

The signal produced in the presence of a protein-protein interaction between the bait and prey proteins should be detectably different than the signal produced in the absence of a protein-protein interaction. By detectably different, it is meant that the signal produced in the presence of a test protein-protein interaction can be higher or lower than the signal produced in the absence of a test protein-protein interaction.

Activation and Repression of Biological Processes

In other preferred embodiments, the reporter system comprises a polypeptide that activates or represses a biological process, and optionally further comprises a localization polypeptide. It is recognized that the polypeptide which activates or represses a biological process may also be the bait and/or prey polypeptide of interest. In the present embodiment, a bait-prey interaction is detected by the activation or repression of a biological process resulting from the bait-prey interaction. Activation may result from targeting activation factors to their sites of action by using a bait-localization fusion protein and prey-activation fusion proteins for example. Activation may also result from sequestering repression factors from their intended sites of action. Repression of a biological process to detect bait-prey interaction may result from sequestering repression factors from their intending sites of action to activate a detectable biological process. Repression of a biological process may also result from sequestering activation factors from their intended sites of action to downregulate a detectable biological process.

In a particular preferred embodiment, the reporter system comprises a polypeptide that activates or represses a biological process such as transcription of a reporter gene, a signal transduction pathway, apoptosis pathway, DNA replication, formation of cellular macrostructures (such as the cytoskeleton, nuclear scaffold, mitotic spindle, nuclear pores, centrosomes, and kinetochores), enzymatic processes (such as proteases and kinases), senescence, cell cycle arrest, cell cycle checkpoints, secretion of proteins and molecules, metabolic pathways, translation of RNA, resistance to antibiotics, resistance to viral infection, resistance to chemicals, temperature sensitivity, tolerance or sensitivity to environmental conditions (such as pH, light, radiation, magnetism, desiccation, ionic strength, and pressure), sensitivity or tolerance to cell-cell contact, auxotrophic requirements, endocytosis, and binding of molecules to receptors.

As a non-limiting example of the use of transcription factors to detect bait-prey interactions, a transcription factor may be expressed as the bait or prey protein. A transcription factor may be also expressed as a fusion protein with a bait or prey polypeptide. Non-limiting examples of transcription factors include SREBP (Brown et al. Proc. Natl. Acad. Sci. USA 96:11041-48, Sept. 1998), VP16, GAL4, GCN4, ARD1, B42, NF-KB, p65, Fos, Jun AP-1, p53, Sp1, TFIID, TFIIA, TFIIB, and TBP. Preferably, the transcription factor binds to the promoter of a reporter gene and specifically activates only the reporter gene. In the presence of a bait-prey interaction, the transcription factor is sequestered or hindered from activating a reporter gene. Therefore, the absence of expression of the reporter gene indicates a bait-prey interaction.

Use of transcription factors and reporter genes in accordance with the present invention includes assaying factors and small molecules that disrupt a known protein-protein interaction expressed as the bait-prey pair. In this example, a factor or molecule that disrupts a bait-prey interaction releases the transcription factor to activate a reporter gene indicating the disruption of the bait-prey interaction.

In another preferred embodiment, a transcriptional repression factor is used to detect baitprey interactions. The repressor may be expressed as the bait or prey protein. A repressor factor may be also expressed as a fusion protein with a bait or prey polypeptide. Non-limiting examples of repressor factors include CRO repressor, phage 434 repressor, Lac repressor, artificial zinc-fingers (Beerli et al. Proc Natl Acad Sci U S A 97(4): 1495-500, 2000; Kim and Pabo. J Biol Chem 272(47):29795-800, 1997; Nagaoka et al. J Inorg Biochem 82(1-4):57-63, 2000), the Gro protein of Drosophila (Chen et al. Gene 249(1-2):1-16, 2000), the mammalian protein, RYBP (Garcia et al. EMBO J. 18:3404-3418, 1999), the human protein ACR1 (Kropotov et al. Eur. J. Biochem. 260:336-346, 1999), the human protein ZFM1 (Zhang et al. J. Biol. Chem. 273:6868-6877, 1998), the human homeodomain protein EVX1 (Briata et al. FEBS Lett. 402:131-135, 1997), and Msx2 (Semenza et al. Biochem. Biophys. Res. Commun. 209:257-262, 1995). Furthermore, in the yeast Saccharomyces cerevisiae, association of the histone deacetylase, RPD3, with the transcriptional repressors SIN3 and UME6 results in repression of reporter genes containing the UME6-binding site (Rundlett et al. Nature 392(6678):831-5, 1998). This system may be used as a reporter system in accordance with the present invention.

In another preferred embodiment, a signal-producing polypeptide activates a cellular pathway that produces a detectable product. Such pathways include signal transduction. As a non-limiting example, the SOS (son of sevenless) factor is protein which activates the Ras signaling pathway when targeted to the plasma membrane in the vicinity of Ras (Aronheim et al. Cell 78:949-61, 1994). SOS therefore can be used in the present invention as a signal producing protein which produces a detectable product. In the present embodiment, a bait polypeptide is localized to the plasma membrane in the vicinity of Ras using Ras or another plasma membrane polypeptide. Prey polypeptides are fused to SOS and after expression in cells in accordance with the present invention, are assayed for an interaction with the bait polypeptide. The presence of a bait-prey interaction recruits the SOS protein to the Ras signaling cascade and activates the pathway. The result is phosphorylation of numerous factors/proteins in the pathway. These phosphorylated proteins include MEK, MAPK, Fos, Jun and Myc proteins. The activation of the factors in the pathway therefore results in phosphorylation of amino acid residues which can be detected. For example, bis-phosphorylation at a specific TEY tripeptide sequence in the Erk1/Erk2 MAPK can be detected by antibodies directed against the pTEpY phosphorylated sequence (New England Biolabs, Beverly, Mass.). Thus a detectable signal in the present embodiment is a labeled primary or secondary antibody that binds to a phosphorylated factor in the Ras activation pathway which is not phosphorylated in the absence of membrane bound SOS. The primary or secondary antibodies can be conjugated with a signal producing moiety such as a radioisotope or a chemiluminescent moiety such as horseradish peroxide using luminol as a substrate.

In yet another preferred embodiment of detection, two signal producing polypeptides may be used to detect an interaction between polypeptides fused to each signal producing polypeptide, where one signal producing polypeptide is a light emitting protein and another signal producing polypeptide is a transcription factor that is activated by the emitted light. A particularly preferred detection system utilizes the White collar proteins (WC-1 and WC-2) and the albino promoter (Talora et al. EMBO. 18:4961-68, 1999; DeFabo et al. Plant Physiol. 57:440-445, 1976). WC1 and 2 are activated by blue light which triggers a series of phosphorylation events. The phosphorylated white collar proteins are capable of binding to the albino 3 (al-3) promoter to cause induction of a reporter gene (Macino et al. Mol. Cell. Biol. 9(3):1271-6, 1989; Carattoli et al. Mol Microbiol. 13(5):787-95, 1994; Linden and Macino. EMBO. 16:98-109, 1997; Carattoli et al. Genetics. 92:6612-6616, 1995).

To detect protein-protein interactions in accordance with the present invention, a first polypeptide (bait) is fused to a blue-emitting GFP mutant. The emission wavelength of the blue GFP mutant should not activate the WC proteins. A second polypeptide (prey) is fused to a WC protein (WC-1 or WC-2). A reporter gene is used which contains the albino-3 (al-3) promoter upstream of the gene. When two polypeptides being assayed are physically interacting, the interaction brings the blue GFP into close proximity to a WC protein. The blue emission from the blue GFP mutant activates the WC by triggering a series of phosphorylation events to enable the WC protein to bind to the albino-3 promoter and thus activating expression of the reporter gene operatively-linked to the albino-3 promoter. Expression of the reporter gene therefore indicates an interaction between the bait and prey fusion proteins.

In yet another embodiment, a signal-producing polypeptide is a polypeptide that can bind to a fluorescent compound through electrostatic (e.g. biotin-streptavidin) or covalent interactions. One particularly preferred compound is FLASH-EDT2 (4′,5′-bis(1,3,2-dithioarsolan-2-yl)fluorescein) which fluoresces when bound to a protein (Griffin et al. Science 281:269-272, 1998). FLASH-EDT2 is a membrane permeable compound that binds to a tetracysteine containing helix and fluoresces when bound. In accordance with the present invention, a peptide capable of binding FLASH-EDT2 is fused to either a bait protein or a prey protein (e.g. a polypeptide containing the proper orientation of four Cys residues in an alpha helix (i, i+1, i+4, and i+5)). Detection of a bait-prey interaction may be performed using FRET or BRET according to the present teaching. Detection of a bait-prey interaction may also be performed using a localization polypeptide fused to either the bait or prey protein, where the presence of a bait-prey interaction sequesters the fluorescent compound within the cell to provide a detectable signal.

### EXAMPLES

Those of ordinary skill based on the teachings herein can readily construct expression vectors containing promoters, multiple cloning sites for inserting libraries or coding sequences of interest, and IRES sequences for each additional coding sequence according to the present teachings using common methods in molecular biology (see Ausubel et al. supra).

Particularly preferred expression vectors of the present invention are constructed having the preferred structure/sequence:

5′-LTR-promoter-MCS-detection-IRES-MCS-detection-LTR-marker-3′

Preferred inventive expression constructs include one or more Long Terminal Repeats (LTR). LTRs are used to direct stable integration of the sequence into the host genome. In addition, preferred constructs may further include one or more multiple cloning sites (MCS).

MCS have multiple restriction enzyme recognition sites which allow the insertion of nucleic acids using a variety of commercially available restriction enzymes. In certain preferred embodiments, the detection coding sequence encoding the detector polypeptide is positioned in frame with the coding sequences to result in proper translation. It is recognized that the detection sequence can be 5′ or 3′ to the coding sequences that are inserted into the MCS. It is also recognized that according to the present teachings, detection sequences may be fused to one or both members of the bait-prey pair being assayed.

As generally described above, selection markers are used to select cells that are expressing the construct. Markers are any selectable markers and include antibiotic resistance markers such as amp^{r}, tet^{r}, hyg^{r}, and neo^{r}.

In the present example, a single nucleic acid molecule encodes a pair of bait-prey proteins to be assayed from an interaction, and is transfected into the host cell line by a retrovirus at a low multiplicity of infection (MOI) such that individual cells are infected by at most one virus particle. Standard transfection conditions for a low MOI are readily known to those skilled in the art and may vary slightly depending upon the host cells used, the viral strain used and the protocol used. The proper transfection conditions are thus easily determined without undue experimentation (see Gu et al. J. Biol. Chem. 275:9120-9130, 2000 incorporated by reference). For a general reference of protocols, see Ausubel et al. (supra). It is recognized that experimental error may result in slightly more than the desired number expressed. However, statistically at low MOI, a majority of cells will not be infected, and a minority of cells will be singly infected.

One non-limiting example of a construct using two promoters to express two polypeptides from one construct for use in accordance with the present invention has the structure:

5′-CMV promoter-MCS-yellowFP-SV40polyA-----EF1alpha promoter-MCScyanFP-SV40polyA-----resistance marker-pUCori-3′

Use Of Multiple Vectors

In another embodiment of the present invention, bait proteins are expressed by a first viral expression vector, and prey proteins are expressed by a second viral expression vector, where the first and second expression vectors contain different selectable markers. The bait protein(s) may be a specific protein of interest or alternatively may be encoded by a library of genes. The prey protein(s) may also be a specific protein of interest or may be encoded by a library of genes.

Nucleic acids encoding bait and prey proteins are introduced in a collection of cells such that individual cells express a single bait-prey combination preferably using viral-mediated nucleic acid transfection. The expression vectors are packaged into the viruses which can be the same viral line or different viral line using standard techniques. The packaged viral particles containing coding sequences for bait and prey proteins are transfected into a collection of host cells at a low multiplicity of infection such that individual cells in the collection are transfected by at most two virions. The transfection of the first viral line containing bait coding sequences and the second viral line containing prey coding sequences can be performed simultaneously or serially. Cells that are doubly transfected with viruses containing coding sequences for bait proteins and prey proteins are selected using the two different selectable markers for each expression vector. A majority of cells will be transfected with one virus (or no viruses) containing one expression vector and will not survive a double selection process. A small percentage of cells will be doubly transfected. Only cells that are doubly transfected and contain one vector from each library will survive a double selection process. Those of ordinary skill in the art are readily capable of determining the proper transfection conditions at the proper low multiplicity of infection without undue experimentation (Ausubel et al., supra).

Double selection of the transfected cells ensures that only cells that survive are transfected by two virions each containing a different selectable marker. The result is that cells that survive double-selection contain coding sequences to express a single bait-prey polypeptide combination. The bait-prey polypeptides are then assayed for an interaction using a reporter system in accordance with the present invention.

In one preferred embodiment, a physical interaction between a bait protein expressed from a first vector and a prey protein expressed from a second vector is detected by BRET in accordance with the present invention. The first or second vectors may contain coding sequences from a library such that the bait is a protein of interest to be screened for an interaction against a library of prey proteins. Alternatively or additionally, the first and second vectors may both encode a library of proteins to be screened against each other for novel protein-protein interactions. The library of proteins may be encoded by the same library or by different libraries.

To provide illustrative details regarding constructs which may be used for BRET, but without limitations to only these constructs, the following description depicts the elements of constructs and their relative order which may be used. The GFP fusion encoding construct utilizes a CMV promoter and zeocin marker with a pUC origin of replication site (pUCOri).

5′-CMV-MCS-GFP-(SV40polyAdenylation)-zeocin-pUCori-3′

The luciferase fusion encoding construct also uses a CMV promoter with a kanamycin and/or zeocin marker:

5′-CMV-MCS-luciferase-(SV40polyAdenylation)-Kan/zeocin-3′

Proteins of interest and/or libraries of interest are cloned into the MCS using standard methods. Constructs are introduced into a cell line of interest in accordance with the present invention. Expressed proteins are then assayed for interactions by BRET. If an interaction is detected between two or more proteins, then the constructed encoding the interacting proteins may be isolated and sequenced to identify the interacting proteins.

The following examples are not meant to limit the scope of the present invention to only these embodiments, but rather are meant to provide experimental and illustrative details to those skill in the art to practice the invention. Those of ordinary skill in the art are readily aware of alternative methods and reagents to be used to practice the present invention without undue experimentation while still using the claimed reagents and steps of the claimed methods. Those of ordinary skill in the art are also readily aware that routine experimentation may be necessary to practice the claimed invention after reading the present description and the references cited, which are all incorporated herein by reference as if each reference were individually incorporated by reference.

We claim:

1. A method of assaying protein-protein interactions comprising steps of:
(a) providing a collection of host cells;
(b) providing at least one library of nucleic acids encoding proteins to be assayed for an interaction with each other;
(c) introducing the nucleic acids into the host cells, wherein a resulting collection of host cells comprises individual host cells containing coding sequences which encode a single set of proteins to be assayed for an interaction;
(d) incubating the resulting collection of host cells under conditions that allow expression of the single combination of proteins to be assayed; and
(e) determining if the proteins in the set interact, wherein if an interaction is detected, then identifying the proteins in the set.

2. A method of screening a library for protein-protein interactions with a protein of interest comprising steps of:
(a) providing a collection of host cells;
(b) providing a library of nucleic acids encoding proteins to be assayed for an interaction, and further providing nucleic acids encoding a protein of interest;
(c) introducing the library of nucleic acids and the nucleic acids encoding the protein of interest into the host cells, wherein a resulting collection of host cells comprises individual host cells containing one coding sequence from the library and the coding sequence for the protein of interest;
(d) incubating the resulting collection of host cells under conditions that allow expression of the coding sequence from the library and expression of the coding sequence for the protein of interest; and
(e) determining if the proteins in the set interact, wherein if an interaction is detected, then identifying the proteins in the set.

3. A method for assaying protein-protein interactions comprising the steps of:
(a) providing a collection of eukaryotic cells;
(b) providing at least one library of nucleic acids encoding proteins to be assayed for an interaction, wherein each nucleic acid molecule contains coding sequences for a single combination of proteins to be assayed;
(c) introducing the nucleic acids into the host cells, wherein individual cells are transfected with at most a single nucleic acid molecule from the library;
(d) incubating the resulting collection of host cells under conditions that allow expression of the single combination of proteins to be assayed; and
(e) determining if the proteins in the set interact, wherein if an interaction is detected, then identifying the proteins in the set.

4. A method for assaying protein-protein interactions comprising the steps of:
(a) providing a collection of eukaryotic cells;
(b) providing at least one library of nucleic acids encoding proteins to be assayed for an interaction with the protein of interest also encoding a protein of interest, wherein each nucleic acid molecule contains a coding sequence for the protein of interest and further contains a coding sequence from the library;
(c) introducing the nucleic acids into the host cells, wherein individual cells are transfected with at most a single nucleic acid molecule from the library;
(d) incubating the resulting collection of host cells under conditions that allow expression of the single combination of proteins to be assayed; and
(e) determining if the proteins in the set interact, wherein if an interaction is detected, then identifying the proteins in the set.
